(19) United States
(12) Patent Application Publication
(10) Pub. No.: US 2004/0241848 A1
(43) Pub. Date: Dec. 2, 2004

(54) Method of detecting microorgan ISMS using labelled electron acceptors

(75) Inventor: Hyo-bong Hong, Gyeonggi-do (KR)

Correspondence Address: BURNS DOANE SWECKER & MATHIS L L P, POST OFFICE BOX 1404, ALEXANDRIA, VA, 22313-1404, US

(73) Assignee: Samsung Electronics Co., Ltd., Gyeonggi-do (KR)

(21) Appl. No.: 10/847,998

(22) Filed: May 19, 2004

(30) Foreign Application Priority Data
May 28, 2003 (KR) 10-2003-0034031

(51) Int. Cl. C12Q 1/16; C12N 5/00; C12N 5/02

(52) U.S. Cl. 435/383

(57) ABSTRACT

Provided is a method of detecting microorganisms. The method includes: loading a contaminant sample in a sealed chamber; replacing air in the sealed chamber with air containing isotopically labelled electron acceptors followed by incubation; and comparing the content of metabolites of the electron acceptors for the contaminant sample with that for a standard sample containing no microorganisms, based on isotopic analysis of the air in the sealed chamber.

### BACKGROUND OF THE INVENTION

This application claims priority from Korean Patent Application No. 2003-34031, filed on May 28, 2003, in the Korean Intellectual Property Office, the disclosure of which is incorporated herein by reference in its entirety.

1. Field of the Invention

The present invention relates to a method of detecting microorganisms using isotopically labelled electron acceptors.

2. Description of the Related Art

As electronic, information technology (IT), and precision machine industries are rapidly in progress, demand on nanometer level (sub micron) devices is being increased. Accordingly, even a slight trace contaminants produced during a process may pose a serious problem for the overall process. In particular, with respect to contamination with organisms such as microorganisms, biomass may increase with time, and thus, previous analysis and removal of microorganism contamination are essential.

However, it is very difficult to determine whether the trace contaminants produced in fabrication of precision electronic devices such as semiconductors and liquid crystal displays (LCDs) where a clean environment is an absolute requirement, are organic materials or microorganisms. Further, a measurement of microorganism activity may be very difficult. Generally, gas chromatography/mass spectrometry (GC/MS) has been used to measure the activity of trace contaminants. For example, GC/MS has been used for applications such as the analysis of air pollutants, water contaminants, and residual agricultural chemicals in the soil.

Various biological methods including collection and analysis for a relatively high number of microorganisms have been developed. However, contaminant sample collection in electronic devices, etc. where a clean environment is an absolute requirement is difficult due to a very low concentration of microorganisms, which renders application of a common biochemical method difficult and increases the duration of contaminant analysis.

Analysis of contaminants, including microorganisms, by general GC/MS or liquid chromatography/mass spectrometry (LC/MS) has been focused on direct analysis of biological substances or analysis of metabolites of microorganisms. Here, the biological substances include proteins, DNAs, and hydrocarbons. In this case, however, it is hard to collect contaminant samples from contamination sites. Even though contaminant samples are collected and analyzed, it is difficult to determine whether microorganisms have biological activity. Furthermore, even though respiratory metabolites are identified by GC/MS, it is impossible to determine whether the metabolites are derived from the microorganisms or surrounding environments, which makes it difficult to ascertain the presence of biological activity of the microorganisms.

In this regard, the present inventor found a prompt and accurate method for ascertaining the presence of biological activity of microorganisms by metabolite analysis and completed the invention.

### SUMMARY OF THE INVENTION

The present invention provides a prompt and accurate method for detecting microorganisms with biological activity.

According to an aspect of the present invention, there is provided a method of detecting microorganisms, comprising: loading a contaminant sample in a sealed chamber; replacing air in the sealed chamber with air containing isotopically labelled electron acceptors followed by incubation; and comparing the content of metabolites of the electron acceptors for the contaminant sample with that for a standard sample containing no microorganisms, based on isotopic analysis of the air in the sealed chamber.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates the analytic result of C¹⁸O₂ for a standard sample containing no microorganisms;

FIG. 2 illustrates the analytic result of C¹⁸O₂ for a sample containing microorganisms;

FIG. 3 illustrates the analytic result of C¹⁸O₂ for samples containing varying concentrations of microorganisms; and

FIG. 4 illustrates a chamber used in the present invention and a sequential process of detecting microorganisms using the chamber.

### DETAILED DESCRIPTION OF THE INVENTION

The present invention provides a method of detecting microorganisms, including: loading a contaminant sample in a sealed chamber; replacing air in the sealed chamber with air containing isotopically labelled electron acceptors followed by incubation for a predetermined time; and comparing the content of metabolites of the electron acceptors for the contaminant sample with that for a standard sample containing no microorganisms, based on isotopic analysis of the air in the sealed chamber.

The microorganisms that can be used herein are not particularly limited and include aerobic and anaerobic microorganisms. The microorganisms may be aerobic bacteria or eukaryotic cells. The microorganisms may also be anaerobic bacteria or eukaryotic cells. Preferably, the microorganisms are Pseudomonas sp., Enterobacteria sp., Bacillus sp., or yeasts, but are not limited thereto.

The electron acceptors may be O₂, NO₃⁻, CO₂, or SO₄²⁻ and the metabolites of the electron acceptors may be CO₂, NO₂⁻, CH₄, or H₂S, but are not limited thereto. Preferably, the electron acceptors are O₂ and the metabolites of the electron acceptors are CO₂. Metabolism of representative electron acceptors in microorganisms is represented as follows:

Carbon source+O₂ →CO₂+H₂O

NO₃⁻+2e⁻+2H⁺→NO₂⁻+H₂O

The isotopic analysis may be performed according to a method commonly used in the pertinent art, for example, GC/MS or LC/MS in an appropriate condition.

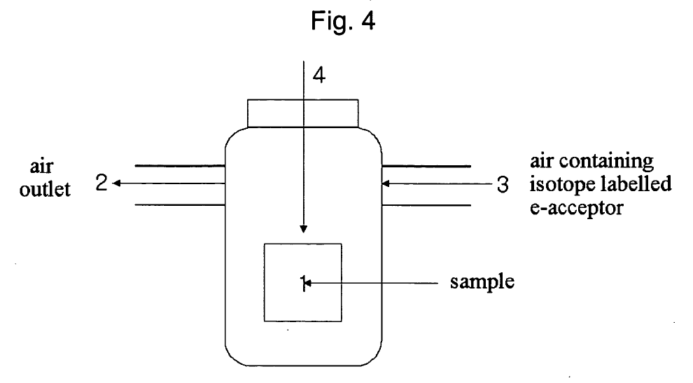

FIG. 4 schematically illustrates a contaminant sample-containing chamber that can be used in the present invention and a sequential process of detecting microorganisms using the chamber.

Referring to FIG. 4, first, a contaminant sample is collected from a clean working environment such as a semiconductor or LCD fabrication plant. After loading the sample into the chamber (Step 1), original air is emitted from the chamber (Step 2) and then the chamber is filled with fresh air containing isotopically labelled electron acceptors, for example, ¹⁸O₂ (Step 3). For this, the chamber may be formed with an inlet valve and an outlet valve. The sample is incubated in the chamber filled with the air containing the isotopically labelled electron acceptors, for example, ¹⁸O₂, for a predetermined time. After the incubation, the content of metabolites, for example, C¹⁸O₂, of the electron acceptors is determined by isotopic analysis of the air by GC/MS, and is compared with that for a standard sample containing no microorganisms to thereby ascertain the presence of microorganisms (Step 4).

Hereinafter, the present invention will be described more specifically by the following Example. However, the following Example is provided only for illustrations and thus the present invention is not limited to or by them.

### EXAMPLE 1

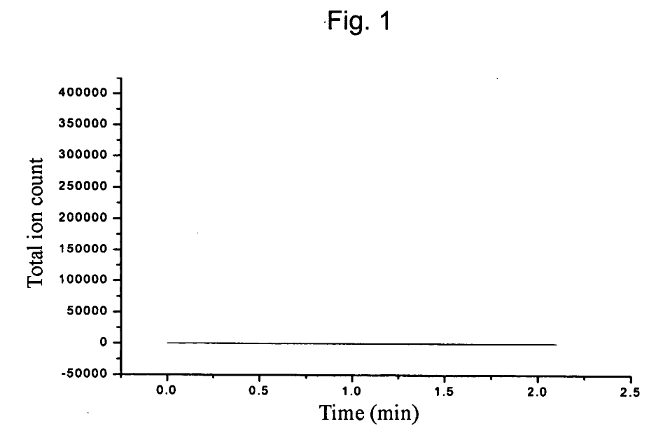

A 20 mM phosphate buffer of a contaminant sample (0.5 ml) containing 100 cfu/ml of bacteria strains, Pseudomonas sp. was loaded into a glass chamber with capacity of 1.5 ml. A head space of the sample was filled with labelled oxygen (¹⁸O)-containing air. Then, the chamber was sealed and the sample in the chamber was incubated at 28° C. for 4 hours. About 0.5 ml of an air sample was then drawn from the chamber using a gas tight syringe and analyzed by GC/MS. The results are shown in FIGS. 1, 2, and 3.

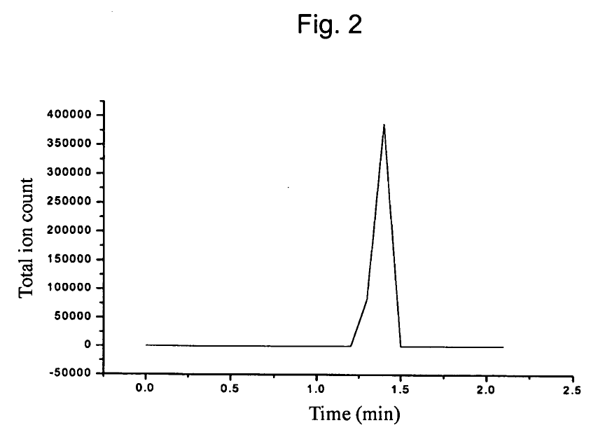

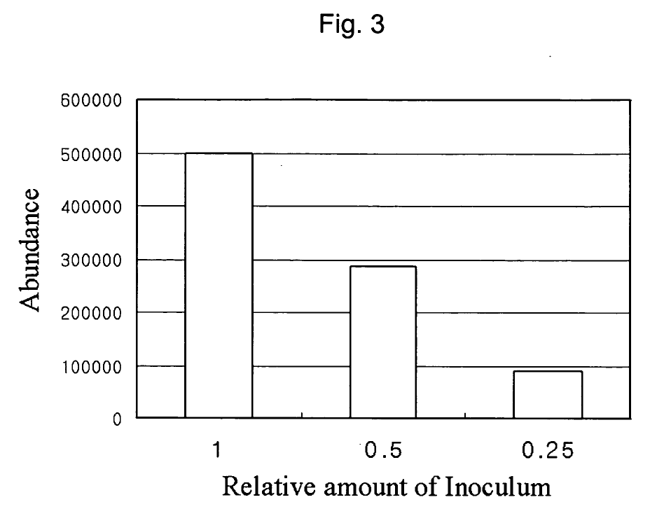

Referring to FIGS. 1 and 2, while no substitution of ¹⁸O occurred in the standard sample containing no microorganisms, O₂ of CO₂ produced in the sample containing the bacteria strains was substituted by ¹⁸O₂. Also, the amount of ¹⁸O₂ incorporated C¹⁸O₂ varied linearly with respect to a change in the amount of the bacteria strains, as shown in FIG. 3. From the above results, it can be seen that the presence of microorganisms in contaminants can be ascertained by incubation of the contaminants in the presence of isotopically labelled electron acceptors.

As apparent from the above descriptions, according to a method of the present invention using isotopically labelled electron acceptors, microorganisms in gas, liquid, or solid samples can be promptly and accurately detected.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of detecting microorganisms, comprising:
loading a contaminant sample in a sealed chamber;
replacing air in the sealed chamber with air containing isotopically labelled electron acceptors followed by incubation; and
comparing the content of metabolites of the electron acceptors for the contaminant sample with that for a standard sample containing no microorganisms, based on isotopic analysis of the air in the sealed chamber.

2. The method of claim 1, wherein the microorganisms are aerobic bacteria or aerobic eukaryotic cells.

3. The method of claim 2, wherein the isotopically labelled electron acceptors are ¹⁸O₂ and the metabolites of the electron acceptors are C¹⁸O₂.
